SEQUENCE LISTING (2 sequences)

SEQ ID NO: 1 - DNA, 834 bp, Vibrio sp.
gcctaccatgcagtcgagcggaaacgagttaactgacccttcgggtgacgttaacggcgtcgagcggcggacgggtgagtaatgcctgggaaattgccctgatgtgggggataaccattggaaacgatggctaataccgcataatgccttcgggccaaagagggggaccttcgggcctctcgcgtcaggatatgcccaggtgggattagctagttggtgaggtaatggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtgtgaagaaggccttcgggttgtaaagcactttcagcagtgaggaaggcgggtacgttaatagcgtgctcgtttgacgttagctgcagaagaagcaccggctaactccgtgccagcagccgcggtaatacggagggtgcgagcgttaatcggaattactgggcgtaaagcgcatgcaggtggttcgttaagtcagatgtgaaagcccggggctcaacctcggaactgcatttgaaactggcggactagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaaacgatgtctacttggaggttgtggccttgagccgtggctttcggagctaacgcgtta

SEQ ID NO: 2 - DNA, 875 bp, Vibrio sp.
gcgcggcctaccatgcagtcgagcggaaacgagttaactgacccttcgggtgacgttaacggcgtcgagcggcggacgggtgagtaatgcctgggaaattgccctgatgtgggggataaccattggaaacgatggctaataccgcataatgccttcgggccaaagagggggaccttcgggcctctcgcgtcaggatatgcccaggtgggattagctagttggtgaggtaatggctcaccaaggcgacgatccctagctggtctgagaggatgatcagccacactggaactgagacacggtccagactcctacgggaggcagcagtggggaatattgcacaatgggcgcaagcctgatgcagccatgccgcgtgtgtgaagaaggccttcgggttgtaaagcactttcagcagtgaggaaggcgggtacgttaatagcgtgctcgtttgacgttagctgcagaagaagcaccggctaactccgtgccagcagccgcggtaatacggagggtgcgagcgttaatcggaattactgggcgtaaagcgcatgcaggtggttcgttaagtcagatgtgaaagcccggggctcaacctcggaactgcatttgaaactggcggactagagtactgtagaggggggtagaatttcaggtgtagcggtgaaatgcgtagagatctgaaggaataccggtggcgaaggcggccccctggacagatactgacactcagatgcgaaagcgtggggagcaaacaggattagataccctggtagtccacgccgtaacgatgtctacttggaggttgtggccttgagccgtggctttcggagctaacgcgttaagtagaccgcctggggagtacggtcgcaagattaaac

(19) United States
(12) Patent Application Publication
(10) Pub. No.: US 2007/0196901 A1
(43) Pub. Date: Aug. 23, 2007

(54) Kahalalide-producing bacteria

(76) Inventors: Russell T. Hill, Baltimore, MD (US); Julie Enticknap, Baltimore, MD (US); Karumanchi V. Rao, Oxford, MS (US); Mark T. Hamann, Oxford, MS (US)

Correspondence Address: INTELLECTUAL PROPERTY / TECHNOLOGY LAW, PO BOX 14329, RESEARCH TRIANGLE PARK, NC, 27709, US

(21) Appl. No.: 10/570,699

(22) PCT Filed: Nov. 1, 2004
(86) PCT No.: PCT/US04/36201

§ 371 Date: Nov. 1, 2006

Related U.S. Application Data
(60) Provisional application No. 60/516,006, filed on Oct. 31, 2003.

(51) Int. Cl. C12P 19/62; C12N 1/21; C12Q 1/68

(52) U.S. Cl. 435/76; 435/252.3; 435/6

(57) ABSTRACT

Disclosed are strains of Vibrio sp bacteria that produce kahalalides or derivatives thereof, methods of isolating said strains, and 16S rRNA sequences useful in identifying kahalalides producing bacteria.

### BACKGROUND OF THE INVENTION

1. Field of Invention

The present invention relates generally to kahalalide compounds and bacteria producing same, and more particularly, to kahalalide compounds produced by Vibrio sp. strains and, methods of growing same for increased production of kahalalides and derivatives thereof.

2. Description of the Related Art

Secondary metabolites isolated from natural sources, predominantly microorganisms and plants, have provided humankind with many of the therapeutic agents currently on the market. Among opisthobranch marine mollusks, (a subclass of Gastropods), members of two orders, the slug-like sea hares and nudibranchs have become frequent targets for biochemical research, since most of them are conspicuous and accumulate secondary metabolites from their algal or invertebrate animal diets. Sacoglossan mollusks of the genus Elysia, in the Family Plakobranchidae, are represented in Hawaii by a number of species and these mollusks have been found to contain a group of compounds, the kahalalides (6, 7). Structures of some known kahalalides are shown in FIG. 1.

The properties, including activity against AIDS Opportunistic Infections (OI), of kahalalide F (KF) has been described (7). KF has been found to act on cell lysosomes, and has been shown to be effective in clinical trials against prostate cancer (5). KF is actively antiviral (HIV-1 EC50=14.2 μM), and it possesses an antifungal activity against the AIDS-opportunistic infection pathogens that is comparable to the activity of the drugs Amphotericin B and Ketoconazole/Rifampin. It is moderately inhibitory of M. tuberculosis (2). KF is now in Phase II clinical trials and has antitumor activity that singles out the cancerous cells of liver, pancreas, prostate, breast, ovaries and melanoma.

Heretofore, the exact origin of KF and the other kahalalides has not been determined. Prior to the discovery by the present inventors, KF had been isolated from both the alga Bryopsis sp. (0.003%) and the mollusk E. rufescens (0.01%), but there was no understanding as to the true source of KF. Because of the limited availability of Elysia for the isolation of KF, attempts have been made to synthesize KF on a laboratory-scale (10, 11). However, the synthesis of such a large molecule is not cost effective because KF is a relatively large peptide of D configuration and other unusual amino and fatty acids, as well as a cyclic moiety. Further, most synthesizing processes are complicated due to the complex chemical structure of the molecules, the inherent production of unwanted by-products and the high cost of production.

Thus, it would be highly advantageous to determine the exact source of kahalalides and an improved method of generating same that overcome the shortcomings of the prior art.

### SUMMARY OF THE INVENTION

The present invention relates to the discovery that kahalalide F and other kahalalides are produced by bacteria. The present inventors, in order to elucidate the true source of KF, undertook a thorough microbiological examination of the Bryopsis sp. and E. rufescens and discovered a KF-producing microbe associated therewith. Although not wanting to be held to a specific theory, the present inventors theorize that the E. rufescens acquires KF-producing microbes from the surface of the Bryopsis and maintains these microbes as symbionts.

Thus, in one aspect the present invention relates to isolated bacteria that generate at least one kahalalide, wherein the bacteria is closely related to Vibrio sp. as shown in FIG. 2. Specific strains, designated Vibrio sp. strain HV10 and HV1, were confirmed to produce KF when grown in pure culture on artificial medium.

In another aspect, the present invention relates to isolated bacteria that produce KF, and preferably the bacteria is a Vibrio sp., and more preferably, the strain is Vibrio sp. strains HV1 and HV10.

In yet another aspect, the present invention to bacteria comprising a 16S rRNA of SEQ ID NO: 1 or SEQ ID NO: 2, or hybridizes thereto, and produces kahalalide F.

A still further aspect of the present invention relates to bacteria comprising a 16S rRNA nucleotide of SEQ ID NO: 1 or SEQ ID NO: 2 or that hybridize to a complement thereof, and produces kahalalide F.

Another aspect of the present invention relates to an isolated Vibrio sp., which produces KF and which comprises a 16S rRNA nucleotide sequence of SEQ ID NO: 1 or SEQ ID NO: 2 or that hybridizes under high or medium stringency conditions to a cDNA thereof.

In yet another aspect of the present invention relates to a method of isolating kahalalide F producing bacteria comprising the steps of:
a) identifying bacteria containing a 16S rRNA nucleotide sequence of SEQ ID NO: 1 or SEQ ID NO: 2 or that hybridizes thereto under high, medium or low stringency conditions;
b) screening bacteria for kahalalide F producing activity and determining the presence of kahalalide F; and
c) selecting those bacteria having kahalalide F producing activity.

Yet another aspect of the present invention relates to an isolated polynucleotide comprising the sequence as set forth in SEQ ID NO: 1 or SEQ ID. 2 and variants thereof. In particular, such a variant polynucleotide sequence will have at least about 80%, more preferably at least about 90%, and most preferably at least about 95% polynucleotide sequence identity to SEQ ID NO: 1 or SEQ ID NO: 2.

A still further aspect of the present invention relates to an isolated polynucleotide fragment comprising at least six contiguous nucleotides of SEQ ID NO: 1 or SEQ ID NO: 2. Still a further preferred embodiment is a polynucleotide fragment comprising from about 10 to about 200 contiguous nucleotides of SEQ ID NO: 1 or SEQ ID NO: 2.

In yet another aspect, the present invention relates to V. mediterranei strains that produce kahalalide F that are easily grown and handled thereby simplifying the isolation of kahalalide F. Economic production of kahalalide F can be achieved by large-scale fermentation of kahalalide F producing bacteria.

Another aspect of the present invention relates to a method for detecting bacteria that produce kahalalide F, the method comprising the steps of:
(a) mixing at least a fragment of SEQ ID NO: 1, SEQ ID NO: 2, or a cDNA complement thereof, with a biological test sample containing nucleic acids from a bacteria suspected of kahalalide F generating ability, to form a resulting mixture;
(b) subjecting the mixture to high stringency conditions such that hybridization will occur between nucleic acids of the biological test sample and the polynucleotide sequence of SEQ ID NO: 1, SEQ ID NO: 2 or the cDNA complement thereof; and
(c) detecting hybridization complexes in the mixture subjected to hybridization conditions, wherein the presence of a hybridization complex correlates with the presence of a polynucleotide consisting essentially of SEQ ID NO: 1 or SEQ ID NO: 2 in the biological test sample.

Once the bacteria are identified it can be screened for kahalalide F producing activity.

Another aspect of the present invention relates to isolating a gene from a kahalalide F producing bacteria that encodes for kahalalide F, amplifying the gene and including same in a vector system for transfecting a non-kahalalide F producing bacteria host cell.

Yet another aspect of the present invention relates to a method for producing kahalalides by fermentation, the method comprising:
a) culturing at least one Vibrio sp. bacterium having kahalalide producing ability in a culture medium suitable for the growth of the bacterium and production of kahalalide; and
b) separating the kahalalide from the culturing medium.

The cost of production of the cancer drug taxol is currently $400,000/kg as opposed to that of penicillamine which is as little as $10/kg. Interestingly taxol is produced from extraction of plant material, while penicillamine is produced through fermentation. Thus, the production of kahalalides by fermentation is a cost-effective method and simplified by the discovery of the present inventors and the Vibrio strains shown herein that produces KF. Kahalalide-producing Vibrio sp. may be easily grown and handled, simplifying the isolation of kahalalides. Economically feasible production of kahalalides can be achieved by large-scale fermentation of kahalalide-producing bacteria.

Other aspects and advantages of the invention will be more fully apparent from the ensuing disclosure and appended claims.

### BRIEF DESCRIPTION OF THE DRAWINGS

FIG. 1 shows structures of different kahalalides

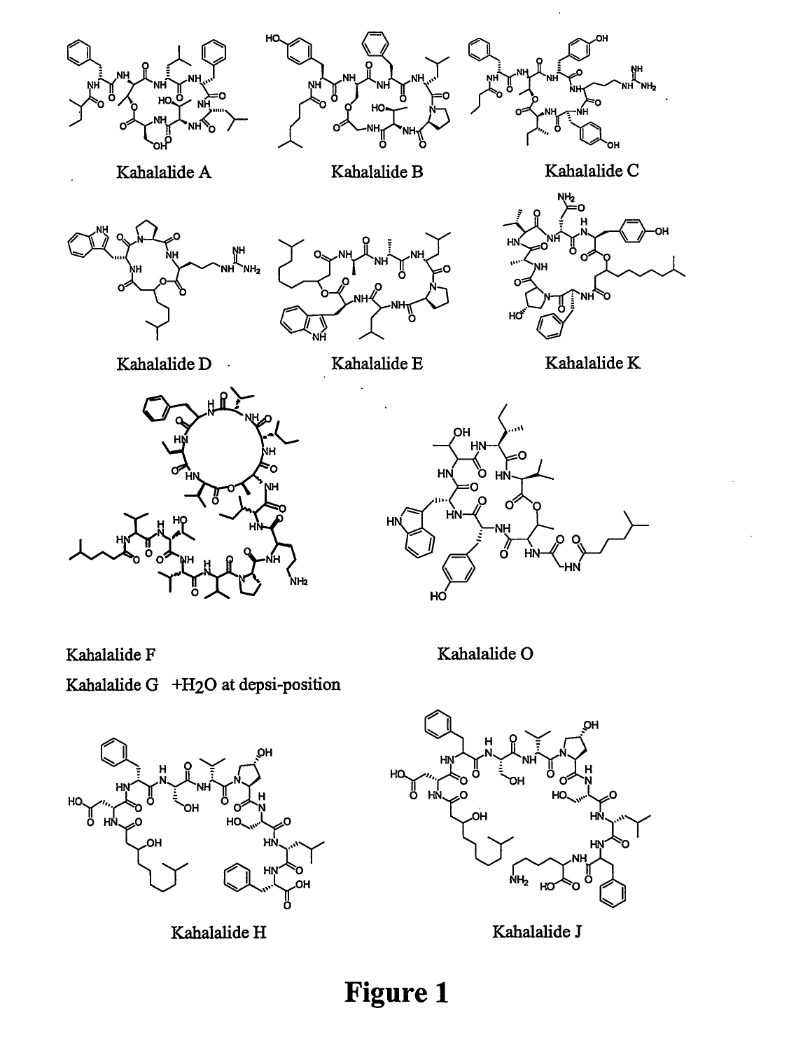

FIG. 2 shows phylogenetic analysis of Vibrio sp. culturable isolates, designated HV, isolated from E. rufescens. Arrows indicate KF-producing strains HV1 and HV10

FIGS. 3 A, B and C show photographs of A) E. rufescens, B) egg mass and C) Bryopsis.

FIGS. 4A and B show culturable colony morphologies from Elysia rufescens (a) and Bryopsis (b).

FIG. 5 shows the HPLC profile of standard KF (signal 2) and test sample (signal 4) from Vibrio sp. stain HV1.

FIG. 6 shows the Mass Spectra of HPLC purified KF from Vibrio sp. strain HV1.

FIG. 7 shows the Proton NMR Spectra of HPLC purified KF from Vibrio sp. strain HV1.

FIG. 8 shows the LCMS Spectra of KF from microbe.

FIG. 9 shows the Mass Spectra of purified kahalalide F from Elysia rufescens.

FIGS. 10A and B show partial 16s rRNA sequence of Vibrio sp. strains HV1 (A) and HV 10 (B).

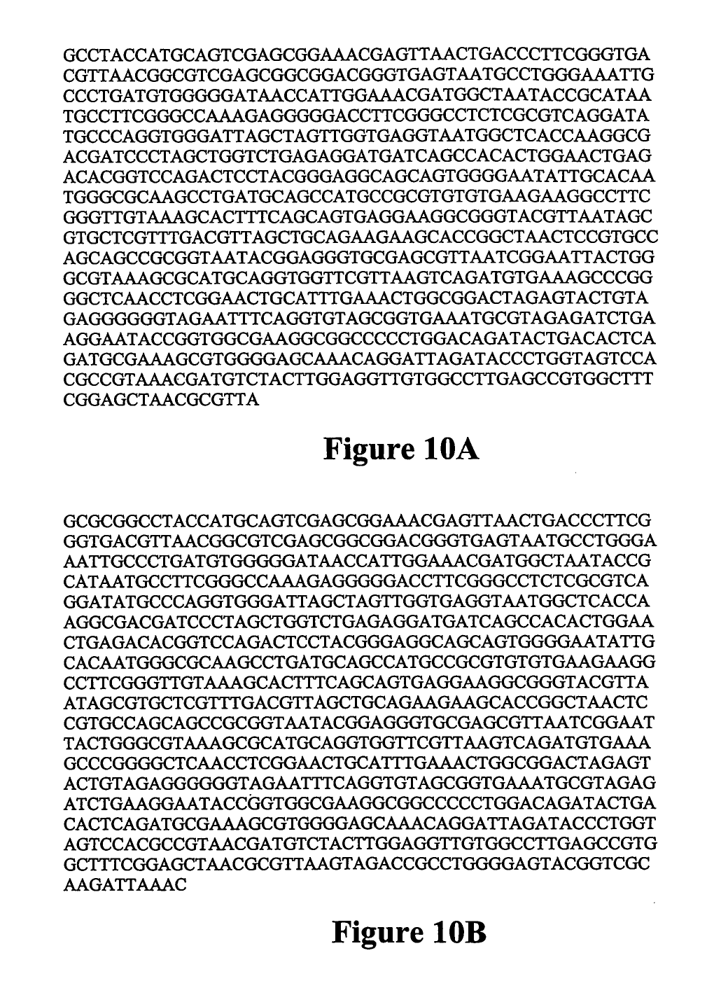

### DESCRIPTION OF PREFERRED EMBODIMENT OF THE INVENTION

Generally, the present invention relates to kahalalides-producing bacteria, methods of isolating kahalalides-producing bacteria, and method of producing kahalalides by culturing bacteria.

The production of kahalalides is simplified by the discovery of Vibrio sp. strains that produce KF. Economic production of kahalalides can be achieved by large-scale fermentation of kahalalide-producing bacteria Identification of Vibrio sp. strains HV1 and HV10 as sources of KF production facilitates cloning of genes involved in KF production, enabling potential KF production in a heterologous microbial host and facilitates genetic manipulation of the pathway in order to produce new kahalalides. Since Vibrio sp. strains HV1 and HV10 produce kahalalide F, it is highly likely that all kahalalides are produced by bacterial strains related to Vibrio sp. strains HV1 or HV10.

In order to facilitate review of the various embodiments of the present invention and provide an understanding of the various elements and constituents used in making and using the present invention, the following terms used in the invention description have the following meanings.

The term “polynucleotide,” as used herein, is a composition or sequence comprising nucleotide subunits, wherein the subunits can be deoxyribonucleotides, ribonucleotides, deoxyribonucleotide analogs, ribonucleotide analogs or any combinations thereof.

The term “variant” as used herein is a nucleic acid sequences with deletions, insertions, or conservative substitutions of nucleotides resulting in a polynucleotide that encodes the same or a functionally equivalent polypeptide.

The term “isolated polynucleotide” as used herein, is a polynucleotide, which is considerably free from naturally occurring cellular components. An isolated polynucleotide would also include the polynucleotide enriched in concentration over its concentration in the cell. Any amplified polynucleotide is defined to be considerably free from cellular components.

The term “nucleic acid sequence” as used herein refers to an oligonucleotide, nucleotide, or polynucleotide, and fragments or portions thereof, and to DNA, cDNA or RNA of genomic or synthetic origin which may be single- or double-stranded, and represent the sense or antisense strand.

The term “hybridization,” as used herein, is defined as when two complimentary strands of two polynucleotides form a double stranded molecule as a result of base-pairing between the individual nucleotides of the two polynucleotides. The two strands of polynucleotides may be completely complimentary, defined as where the two strands of polynucleotides have no corresponding mismatched nucleotide base pairs to the extent of the shortest polynucleotide strand. Alternatively, two strands of polynucleotides may be partially complimentary, defined as where the two strands of polynucleotides have both corresponding matched and mismatched nucleotide base pairs.

The term “hybridization complex,” as used herein, refers to a complex formed between two nucleic acid sequences by virtue of the formation of hydrogen bonds between complementary bases. A hybridization complex may be formed in solution or between a sample polynucleotide sequence present in solution and an variable polynucleotide probe of the present invention immobilized on a solid support (e.g., paper, membranes, filters, chips, pins or glass slides, or any other appropriate substrate to which cells or their nucleic acids have been fixed).

The term “stringency conditions,” as used herein, refers to conditions which permit hybridization between the sample polynucleotide sequences and the variable polynucleotide probe sequences. Suitably stringency conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature.

For applications requiring a high degree of selectivity, relatively high stringency conditions are employed to form the hybridization complexes. For example, relatively low salt and/or high temperature conditions, such as provided by 0.02M-0.15M NaCl at temperatures of 50° C. to 70° C., will be selected. Those conditions are particularly selective, and tolerate little, if any, mismatch between the probe and the template or target strand. Preferably, high stringency hybridization conditions include the following conditions: 6×SSPE, SX Denhardt's reagent, 50% formamide, 42° C., 0.5% SDS, 100 μg/ml sonicated denatured calf thymus or salmon sperm DNA.

Medium stringency hybridization conditions may include the following conditions: 6×SSPE, 5× Denhardt's reagent, 42° C., 0.5% SDS, 100 μg/ml sonicated denatured calf thymus or salmon sperm DNA; and low stringency hybridization conditions may include the following conditions: 6×SSPE, SX Denhardt's reagent, 30° C., 0.5% SDS, 100 μg/ml sonicated denatured calf thymus or salmon sperm DNA.

Formulae for buffers that be used for hybridization in the present invention include: 20×SSPE: 3.6 M NaCl, 0.2 M phosphate, pH 7.0, 20 mM EDTA and water or 50× Denhardt's reagent: 5 g FICOLL Type 400, 5 g polyvinylpyrrolidone, 5 g bovine serum albumin and water.

The term “Kahalalides,” as used herein also includes kahalalides derivatives as recognized in the art, both naturally occurring and synthetic that exhibit similar functionality as known kahalalides.

The term “Polymerase Chain Reaction” and “PCR,” as used herein, refer to a method that results in the linear or logarithmic amplification of nucleic acid molecules. PCR generally requires a replication composition consisting of, for example, nucleotide triphosphates, two primers with appropriate sequences, DNA or RNA polymerase and proteins. These reagents and details describing procedures for their use in amplifying nucleic acids are provided in U.S. Pat. No. 4,683,202 (1987, Mullis, et al.) and U.S. Pat. No. 4,683,195 (1986, Mullis, et al.).

DNA sequence information provided by the present invention relating to strains HV1 and HV10 comprising 16S rRNA nucleotide sequence of SEQ ID NO: 1 (HV1) and SEQ ID NO: 2 (HV10) allows for the preparation of relatively short RNA sequences having the ability to specifically hybridize to nucleotide sequences of other kahalalides producing bacteria. In these aspects, nucleic acid probes of an appropriate length may be prepared based on a consideration of a selected nucleotide sequence. The ability of such nucleic acid probes to specifically hybridize to a 16S rRNA polynucleotide of kahalalide producing bacteria lends them particular utility in a variety of embodiments.

To provide the advantages in accordance with the present invention, a preferred nucleic acid sequence employed for hybridization studies or assays includes probe molecules that are complementary to at least a 10 to 70 nucleotide stretch of a bacteria 16S rRNA, and preferably, a Vibrio sp. strains. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments can be readily prepared by, for example, directly synthesizing the fragment by chemical means, by application of nucleic acid reproduction technology, such as the PCR or by excising selected DNA fragments from recombinant plasmids containing appropriate inserts and suitable restriction enzyme sites.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

In general, it is envisioned that a hybridization probe described herein is useful both as a reagent in solution hybridization as well as in embodiments employing a solid phase. In embodiments involving a solid phase, the test 16S rRNA is adsorbed or otherwise affixed to a selected matrix or surface. This fixed nucleic acid is then subjected to specific hybridization with selected probes under desired conditions. As is well known in the art, the selected conditions depending on the particular circumstances and criteria required (e.g., on the G+C content, type of target nucleic acid, source of nucleic acid, size of hybridization probe). Following washing of the matrix to remove non-specifically bound probe molecules, specific hybridization is detected, or even quantified, by means of the label.

Kahalalides are obtainable by cultivation of bacteria, such as Vibrio sp., and preferably HV1 or HV10 strains. Thus, the present invention further provides a process for the production of kahalalides and derivatives thereof from functionally active bacteria.

The said process comprises cultivation of a culture comprising bacteria, Vibrio sp. under aerobic conditions in a nutrient medium containing one or more sources of carbon, nitrogen and optionally nutrient inorganic salts and/or trace elements, followed by isolation of the said compound and purification in a customary manner.

The nutrient medium preferably contains sources of carbon, nitrogen and nutrient inorganic salts, organic trace elements and optionally other trace elements. The carbon sources are, for example, starch, glucose, sucrose, dextrin, fructose, molasses, glycerol, lactose or galactose, preferably glucose.

The sources of nitrogen are, for example, soybean meal, peanut meal, yeast extract, beef extract, peptone, tryptone, malt extract, corn steep liquor, gelatin or casamino acids, preferably soybean meal and corn steep liquor.

As the organic trace nutrients, amino acids, vitamins, fatty acids, nucleic acids, those containing these substances such as peptone, casamino acid, yeast extract and soybean protein decomposition products are used.

The nutrient inorganic salts and trace elements are, for example, sodium hydrogen phosphate, potassium hydrogen phosphate, sodium chloride, cobalt chloride, calcium chloride, calcium carbonate, potassium nitrate, ammonium sulfate or magnesium sulfate, preferably cobalt chloride and calcium carbonate.

Cultivation of the culture is usually carried out at temperatures between 20-42° C. and in a medium having a pH from about 6.0 to about 8.0, and preferably, about 7.0 to about 8.0. Preferably, the medium is maintained at a pH and salinity value appropriate for growth of the Vibrio sp. bacteria and the production of kahalalides. The fermentation is preferably carried out for about 50 to about 200 hours in order to obtain an optimal yield of kahalalides.

In the resulting culture broth, kahalalides are present primarily in the culture filtrate and can thus be recovered by extraction of the culture filtrate with a water immiscible solvent such as, for example, ethyl acetate, dichloromethane, chloroform or butanol at pH 5-8, or by hydrophobic interaction chromatography using polymeric resins such as DIAION.RTM HP-20 (Mitsubishi Chemical Industries Limited, Japan), AMBERLITE.RTM XAD (Rohm and Haas Industries, U.S.A.), or activated charcoal, or by ion exchange chromatography at pH 5-8.

The crude material can be further purified by using any of the following techniques: normal phase chromatography using alumina or silica gel as stationary phase and eluants such as ethyl acetate, chloroform, methanol or combinations thereof; reverse phase chromatography using reverse phase silica gel like dimethyloctadecylsilylsilica gel, also called RP-18, or diimethyloctylsilylsilica gel, also called RP-8; as stationary phase and eluants such as water, buffers such as phosphate, acetate, citrate (pH 2-8), and organic solvents such as methanol, acetonitrile, acetone, tetrahydrofuran or combinations of these solvents; gel permeation chromatography using resins such as SEPHADEX.RTM LH-20 (Pharmacia Chemical Industries, Sweden), TSKgel TOYOPEARL.RTM HW-40F (TosoHaas, Tosoh Corporation, Japan) in solvents such as methanol, chloroform, acetone, ethyl acetate or combinations of these solvents or SEPHADEX.RTM G-0 and G-25 in water; or counter-current chromatography using a biphasic eluant system made up of two or more solvents such as water, methanol, ethanol, isopropanol, n-propanol, tetrahydrofuran, acetone, acetonitrile, methylene chloride, chloroform, ethyl acetate, petroleum ether, benzene and toluene.

It is contemplated that the present invention may be practiced using either batch, fed-batch or continuous processes and that any known mode of fermentation would be suitable. A classical batch fermentation is a closed system where the composition of the medium is set at the beginning of the fermentation and not subjected to artificial alterations during the fermentation. Thus, at the beginning of the fermentation the medium is inoculated with the desired organism or organisms and fermentation is permitted to occur adding nothing to the system. Typically, however, a batch fermentation is “batch” with respect to the addition of carbon source and attempts are often made at controlling factors such as pH and oxygen concentration. In batch systems the metabolite and biomass compositions of the system change constantly up to the time the fermentation is stopped. Within batch cultures, cells moderate through a static lag phase to a high growth log phase and finally to a stationary phase where growth rate is diminished or halted. If untreated, cells in the stationary phase will eventually die.

A variation on the standard batch system is the fed-batch system. Fed-batch fermentation processes are also suitable in the present invention and comprise a typical batch system with the exception that the substrate is added in increments as the fermentation progresses. Fed-batch systems are useful when catabolite repression is apt to inhibit the metabolism of the cells and where it is desirable to have limited amounts of substrate in the medium. Using a fed-batch system, it is possible to maintain a steady concentration of substrate while accommodating maximum bioconversion of the substrate to product.

Batch and fed-batch fermentations are common and well known in the art and examples may be found in, for example Brock, Thomas D., In Biotechnology: A Textbook of Industrial Microbiology, 2nd ed.; Sinauer Associates, Inc.: Sunderland, Mass., 1989.

Continuous fermentation is an open system wherein a defined fermentation medium is added continuously to a bioreactor and an equal amount of conditioned medium is removed simultaneously for processing. Continuous fermentation generally maintains the cultures at a constant high density. Continuous fermentation allows for the modulation of one factor or any number of factors that affect cell growth or end product concentration. For example, one method will maintain a limiting nutrient such as the carbon source or nitrogen source at low concentration and allow all other parameters to be in excess. In other systems a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

The features and advantages of the invention are more fully shown by the following non-limiting examples.

### EXAMPLE 1

Collection of Mollusks and Green Alga

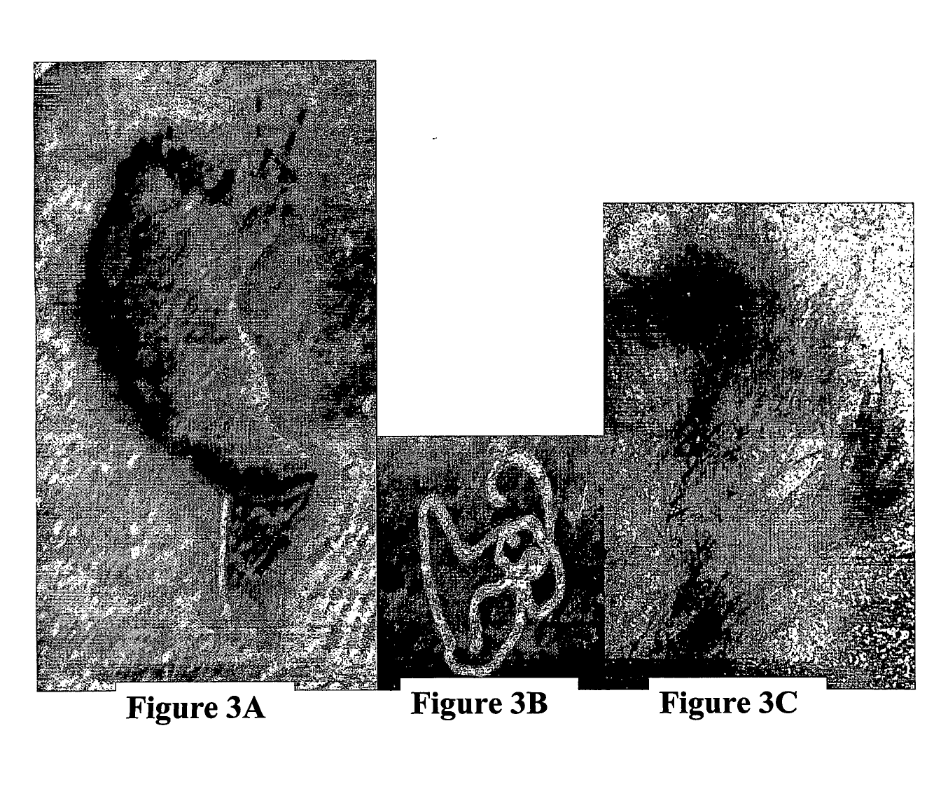

Collection 1. The mollusks E. rufesceus (FIG. 3A) and E. ornata were collected at Black Point, Hi., USA. Egg mass samples from Elysia sp. were collected (FIG. 3B). The green alga Bryopsis sp. (FIG. 3C) was collected at the same site.

Collection 2. The mollusk E. rufesceis was collected at Black Point, Hi., USA. E. rufescens and slime produced by stressed animals were separated and processed for isolation of bacteria.

Samples from collection 1: Three E. rufescens individuals and three E. ornata individuals were processed for microbiology. A section (1 cm³) was cut from the middle of each slug, ground in 10 ml sterile artificial seawater, and 10⁻² and 10⁻³ dilutions were spread onto Marine Agar 2216 (Difco) plates incubated for 3 d at 30 C for isolation of heterotrophic bacteria. Undiluted samples were plated onto ISP Medium 2 Agar (Becton Dickinson) and Starch Casein Agar (10 g l⁻¹ soluble starch, 1 g l⁻¹ casein, 0.5 g l⁻¹ K₂HPO₄, 20 g l⁻¹ NaCl) for isolation of Vibro sp. bacteria.

Each medium was modified to contain 2% (w/v) NaCl and were supplemented with 10 μg ml⁻¹ cycloheximide and 25 μg ml⁻¹ nystatin to control fungal growth and 10 μg ml⁻¹ nalidixic acid to inhibit Gram-negative bacteria. Vibro sp. bacteria isolation plates were incubated at 30 C for 6-9 weeks. An undiluted inoculum (100 μl) was added to Mn+B12 liquid medium (13) for isolation of cyanobacteria Remaining slug tissue was kept frozen at −80 c for DNA extraction.

Two samples of Bryopsis alga were processed following a similar procedure to that described above. Two E. rufescens individuals were dissected and three organs removed from each one were processed for microbiology following an identical procedure.

Samples from collection 2: Two E. rufescens individuals were processed for microbiology by plating on Marine Agar 2216 following the same procedure used during collection 1. In addition, mucus produced by stressed slugs was separated from the slugs and processed by a similar procedure.

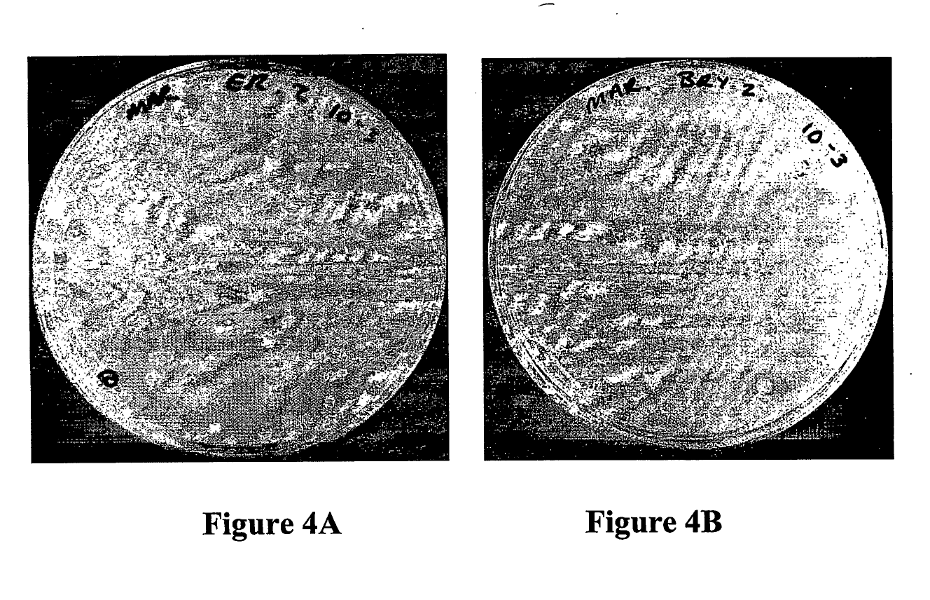

Plate counts of bacteria: Total bacterial counts obtained on Marine Agar 2216 are shown in Table 1. In all cases, isolation plates from E. rufescens and E. ornata were dominated by a single colony morphotype that was present at >95% of the total colony-count as shown in FIG. 4. This morphotype was also present on isolation plates from Bryopsis samples but at low numbers.

Since a single morphotype dominated the Elysia spp. isolation plates, this morphotype was selected for particular attention. A total of 32 isolates of this single morphotype were purified from Collection 1 and kept in cryopreserved stocks. These isolates were designated as listed in Table 2. An additional 96 isolates were obtained in pure culture and cryopreserved from Collection 2.

TABLE 1
Total plate counts from Elysia spp. and Bryopsis samples obtained
on Marine Agar 2216 after 72 h incubation at 30 C.

| Sample | | Colony counts (cfu ml⁻¹) |
|---|---|---|
| Collection 1 | E. rufescens 1 | 2.4 × 10⁴ |
| | E. rufescens 2 | 6.0 × 10⁴ |
| | E. rufescens 3 | 1.6 × 10³ |
| | E. ornata 1 | 4.0 × 10⁴ |
| | E. ornata 2 | 1.4 × 10⁵ |
| | E. ornata 3 | N.D.¹ |
| | Dissected E. rufescens 1 organ 1 | 4.0 × 10⁴ |
| | Dissected E. rufescens 1 organ 2 | 3.6 × 10⁴ |
| | Dissected E. rufescens 1 organ 3 | 9.0 × 10⁴ |
| | Dissected E. rufescens 2 organ 1 | 6.0 × 10⁴ |
| | Dissected E. rufescens 2 organ 2 | 3.0 × 10⁴ |
| | Dissected E. rufescens 2 organ 3 | 6.4 × 10⁴ |
| | Bryopsis 1 | 7.0 × 10⁴ |
| | Bryopsis 2 | 8.0 × 10⁴ |
| Collection 2 | E. rufescens 1 | 4.3 × 10³ |
| | E. rufescens 1 | 2.3 × 10⁵ |
| | Mucus sample | 1.0 × 10⁴ |

¹N.D. Not Determined

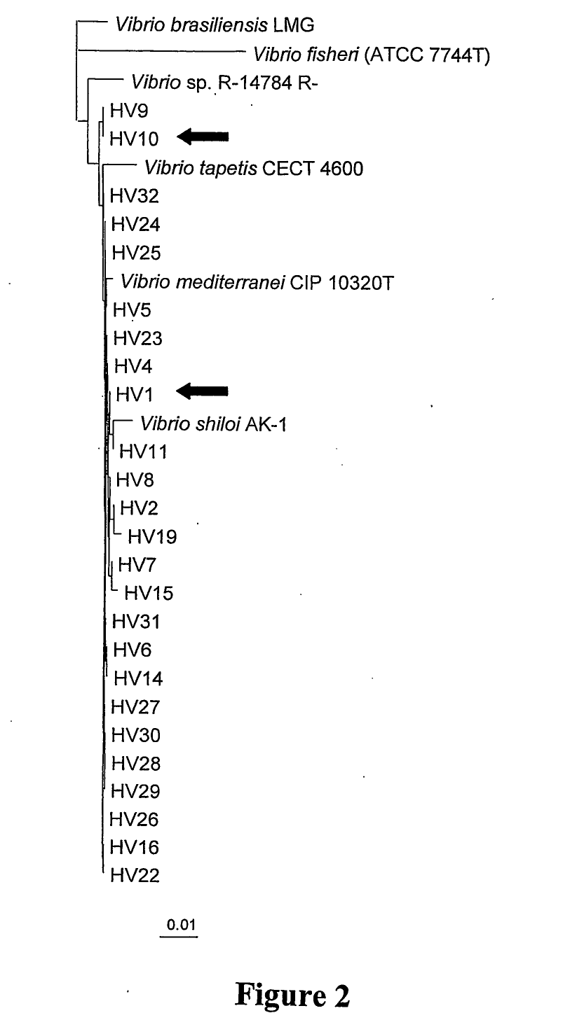

DNA was extracted from the 32 isolates from Collection 1 and these isolates were identified on the basis of 16S rRNA sequence analysis (Table 2 and FIG. 2). The majority of the isolates were identified as strains related to V. mediterranei.

TABLE 2
Source and identification of isolates.

| | | Closest relative |
|---|---|---|
| Isolate | Source of strain | (BLAST analysis) |
| HV1 | E. rufescens, Black Point | V. mediterranei |
| HV2 | E. rufescens, Black Point | V. mediterranei |
| HV3 | E. rufescens, Black Point | Gamma-proteobacterium |
| HV4 | E. rufescens, Black Point | V. mediterranei |
| HV5 | E. rufescens, Black Point | V. mediterranei |
| HV6 | E. rufescens, Black Point | V. mediterranei |
| HV7 | E. rufescens, Black Point | V. mediterranei |
| HV8 | E. rufescens, Black Point | V. mediterranei |
| HV9 | E. rufescens, Black Point | V. mediterranei |
| HV10 | E. rufescens, Black Point | V. mediterranei |
| HV11 | E. ornata, Black Point | V. mediterranei |
| HV12 | E. ornata, Black Point | Marinobacter sp. |
| HV13 | E. ornata, Black Point | Marinobacter sp. |
| HV14 | E. ornata, Black Point | V. mediterranei |
| HV15 | E. ornata, Black Point | V. mediterranei |
| HV16 | E. ornata, Black Point | V. mediterranei |
| HV17 | E. ornata, Black Point | No sequence |
| HV18 | Bryopsis, Black Point | Alpha-proteobacterium |
| HV19 | Bryopsis, Black Point | V. mediterranei |
| HV20 | Egg mass, Black Point | Alpha-proteobacterium |
| HV21 | Egg mass, Black Point | Alpha-proteobacterium |
| HV22 | E. rufescens dissected organ 1 | V. mediterranei |
| HV23 | E. rufescens dissected organ 1 | V. mediterranei |
| HV24 | E. rufescens dissected organ 1 | V. mediterranei |
| HV25 | E. rufescens dissected organ 1 | V. mediterranei |
| HV26 | E. rufescens dissected organ 2 | V. mediterranei |
| HV27 | E. rufescens dissected organ 2 | V. mediterranei |
| HV28 | E. rufescens dissected organ 2 | V. mediterranei |
| HV29 | E. rufescens dissected organ 3 | V. mediterranei |
| HV30 | E. rufescens dissected organ 3 | V. mediterranei |
| HV31 | E. rufescens dissected organ 3 | V. mediterranei |
| HV32 | E. rufescens dissected organ 3 | V. mediterranei |

Identification of isolates: DNA was extracted from the 32 strains listed in Table 2. Strains were unequivocally identified by 16S ribosomal RNA gene sequence analysis as described previously (14). The nearest relative of each isolate was found by BLAST analysis (1) and all isolates were shown to be Vibrio sp. closely related to V. mediterranei and Vibrio shiloi. Phylogenetic trees were then inferred for selected isolates by comparing homologous nucleotides using the neighbor-joining (12), Fitch-Margoliash (4) and maximum parsimony (9) algorithms in the PHYLIP package (3). Evolutionary distance matrices for the neighbor-joining and Fitch-Margoliash methods were generated as described by Jukes and Cantor (8). Tree topologies were evaluated after 1000 bootstrap re-samplings of the neighbor-joining data. The phylogenetic tree for 25 Vibrio strains, including strains HV1 and HV10, is shown in FIG. 2.

Growth of cultures, extraction and purification: Shake-flask culture of Vibrio sp. strains HV1 and HV10 were grown in Marine Broth 2216A for 7 days. A sample of 250 ml fermented broth was centrifuged to separate the material into a cell pellet and supernatant. The supernatant was lyophilized and the residue was extracted three times with EtOH at room temperature. The combined filtrates were evaporated in vacuo and the resultant residue was chromatographed using silica gel flash chromatography (hexane, hexane/EtOAc (1:1), EtOAc, EtOAc/MeOH (9:1; 8:2; 7:3 and 1:1) and MeOH. The fractions obtained from EtOAc/MeOH (8:2) were combined and the solvent was removed by evaporation. Based on the polarity of kahalalides this is the fraction that would typically yield the kahalalides. This fraction was then subjected to HPLC followed by high-resolution mass analysis to determine the presence or absence of KF. Based on the data presented below Vibrio sp. strains HV1 and HV10 clearly yielded KF in a relatively pure form as compared to the peptide mixture from the snail or algae.

HPLC and MS Analysis: Standard KF was purified by reversed phase HPLC, LH20 and normal phase chromatography from Elysia rufescens and compared with the flash chromatography fraction eluted with EtOAc/MeOH; (8:2) from Vibrio sp. strains HV1 and HV10. Both the standard and the EtOAc/MeOH; (8:2) fraction from Vibrio sp. strains HV1 and HV10 were dissolved in methanol and subjected to HPLC. A Prevail C8 5μ, 4.6×250 mm column from Phenomenex was utilized for the analysis. The mobile phase contained water and acetonitrile and the separation was carried out using a linear solvent gradient program beginning with 100% water and ending with 100% CH₃CN over a period of 30 minutes. The flow rate utilized was 1 ml min⁻¹ and the injection volume was 100 μl. The column temperature was kept at room temperature and detection was performed measuring the wavelengths of 214 nm and 254 nm respectively.

HRESI-MS of the samples with comparable retention times was carried out in a positive ion mode on a Bruker FT mass spectrometer equipped with an electrospray ion source and Xcalibur™ data system. Samples were dissolved in methanol at a concentration of 20-200 ng μl⁻¹ and introduced into electrospray needle by mechanical infusion through a micro-syringe at a flow rate of 3 μl min⁻¹.

For all of the analysis extreme care was taken to assure that the samples of Vibrio sp. strains HV1 and HV10 were not contaminated by any KF standard. This includes the use of fresh stationary phases and solvents for chromatography including HPLC columns. In summary none of the chromatography materials utilized in the purification of the standard KF were utilized in the evaluation of the materials from Vibrio sp strains HV1 and HV10.

The culturable bacterial community associated with E. rufescens was found to be dominated by a single morphotype of bacteria and as shown in Table 2 and many of these strains were closely related to V. mediterranei. Two of these strains, designated strains HV1 and HV10, were confirmed to produce KF when grown in pure culture on artificial medium.

### EXAMPLE 2

Detection of KF production by Vibrio sp strains. Four freeze-dried supernatant samples of Elysia-associated Vibrio were evaluated. Each was extracted separately with ethanol at room temperature and purified on Si gel column (VLC) by eluting with hexane, hexane/EtOAc (1:1), EtOAc, EtOAc/MeOH (9:1; 8:2; 7:3 and 1:1) and MeOH. The peptide fraction (EtOAc/MeOH, 8:2) was analyzed for KF and other kahalalides using LCMS. Phenomenex, Luna C8 5μ, 4.6×15 mm column was used for analysis. The mobile phase contained water and acetonitrile and the separation was carried out using a linear solvent gradient program that started with 80% water and decreased to 0% over 60 minutes. The flow rate was 1 ml min⁻¹. The injection volume was 20 μl. From the four samples KF was identified in supernatants from Vibrio sp. strains HV1 and HV10.

### EXAMPLE 3

Additional samples were analyzed from supernatants of two separate scale-up batches of Vibrio sp strains HV1 and HV10 with batches designated 110603 and 102903, respectively. Both the batches were processed and analyzed separately.

Analysis of 110603: Extraction and Purification. The freeze-dried supernatant (126 g) was extracted three times with EtOH at room temperature. The combined filtrates were evaporated in vacuo and the resultant residue was chromatographed using silica gel vacuum liquid chromatography (hexane, hexane/EtOAc (1:1), EtOAc, EtOAc/MeOH (9:1; 8:2; 7:3 and 1:1) and MeOH. Fractions obtained from EtOAc/MeOH (8:2) were combined and the solvent was removed by evaporation. The residue thus obtained was purified by HPLC and subjected to HPLC, MS and LCMS followed by NMR analysis to identify the presence of KF.

HPLC Purification: The peptide-containing fraction was purified by HPLC using acetonitrile-water gradient (with a 22×250 mm C8 column, 254 nm). The peptide fraction obtained by HPLC contained KF and some other impurities as well. The impurities were removed by HPLC—using a 10×250 mm C8 column, acetonitrile-water gradient.

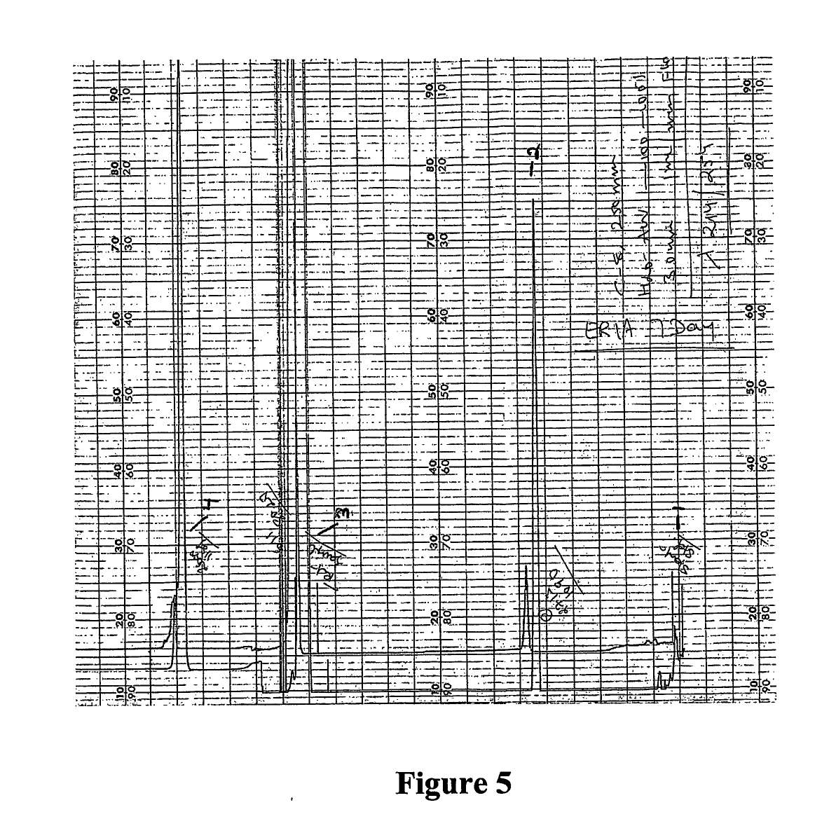

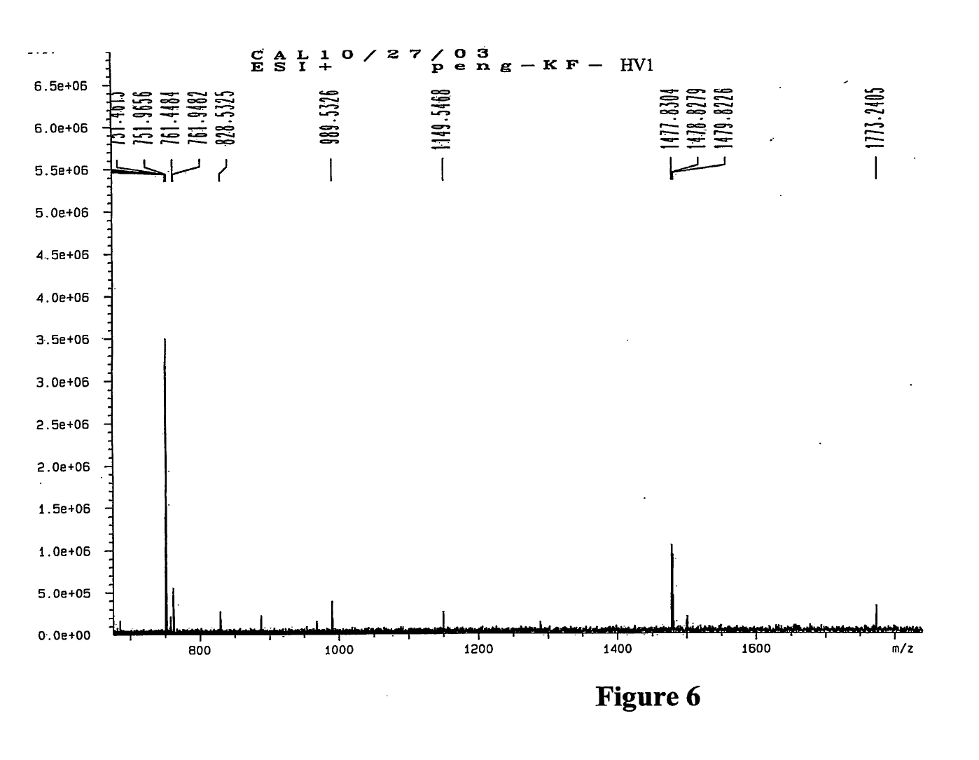

HPLC and MS Analysis. Solutions of standard KF and the test KF fraction (HPLC) were prepared in acetonitrile and subjected to HPLC (FIG. 5). Prevail C8 5μ, 4.6×250 mm column was used for analysis. The mobile phase contains water and acetonitrile and the separation was carried out using a linear solvent gradient program that started with 80% water and decreased to 0% over 30 minutes. The flow rate was 1 ml min⁻¹. The injection volume was 20 μl. The column temperature was kept at room temperature. Detection was performed with wavelength of 214 nm and 254 nm respectively. HRESI-MS of the sample (eluate) was carried out in positive ion mode on a mass spectrometer equipped with an electrospray ion source and an Xcalibur™ data system (FIG. 6). Sample was dissolved in methanol at a concentration of 20-200 ng μl⁻¹ and introduced into electrospray needle by mechanical infusion through a micro-syringe at a flow rate of 3 μl min⁻¹.

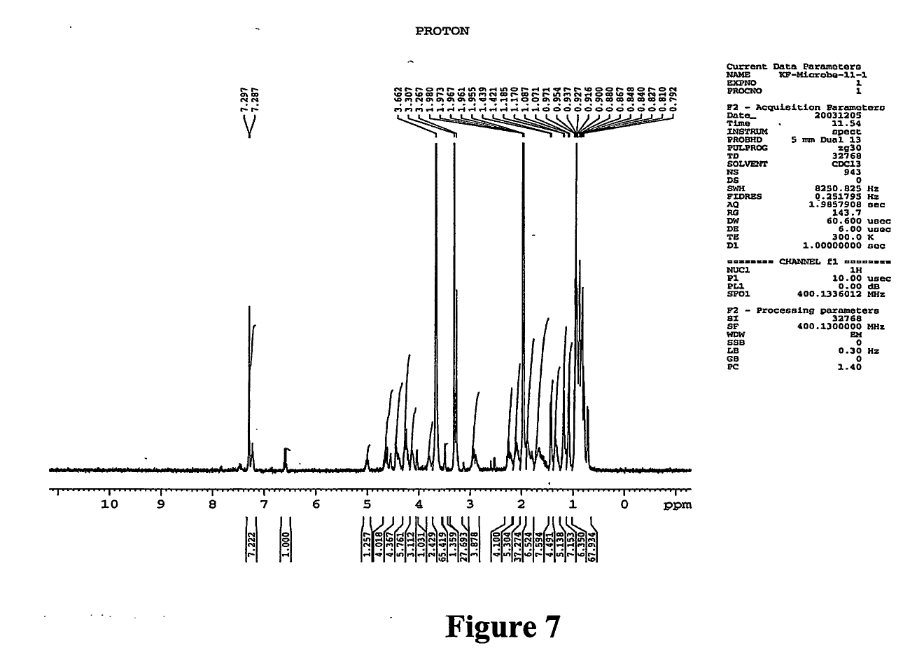

¹H NMR. Shown in FIG. 7 is the proton NMR spectrum recorded in MEOD, for KF obtained from the Vibrio sp. strain HV1 providing extremely solid evidence for the microbial production. The naturally occurring KF exists in a 1:4 ratio of the structure shown earlier and an isomerically branched fatty acid. The material isolated from the Vibrio sp. exists in a significantly different isomeric ration providing further evidence for microbial production.

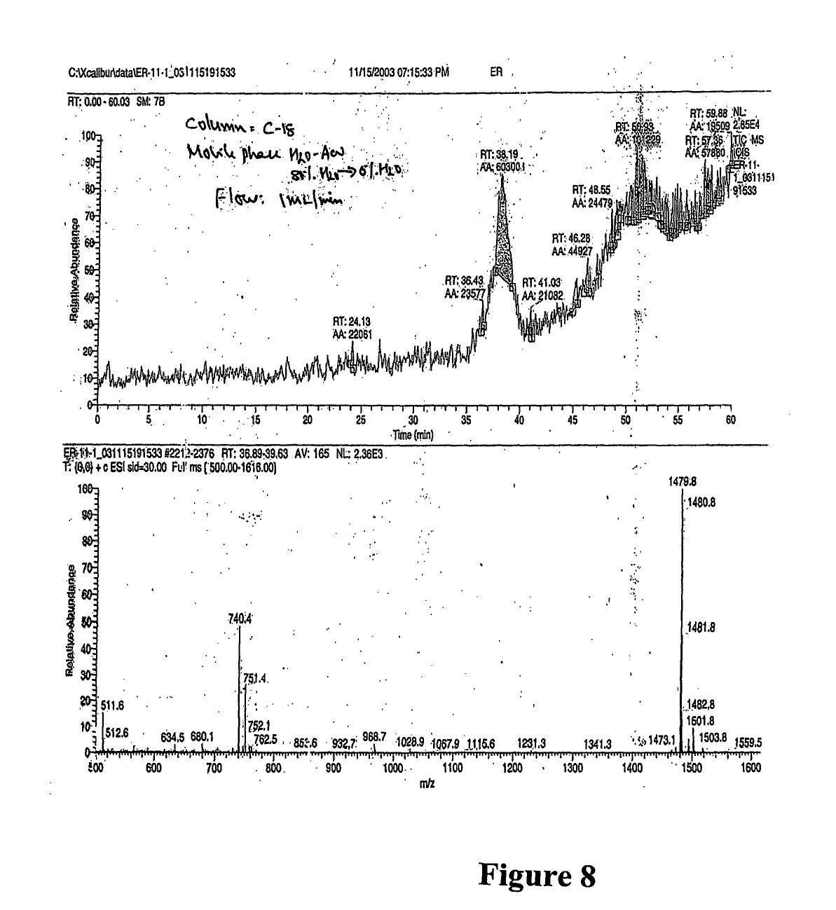

LCMS Analysis. Solutions of standard KF and the test KF fraction (HPLC) were prepared in acetonitrile and subjected to LCMS using reverse-phase C18 column (Waters, 5μ, 4.6×15 mm) eluting at 1.0 ml min⁻¹ flow rate with 60 minute linear gradient from 20% to 100% acetonitrile (FIG. 8).

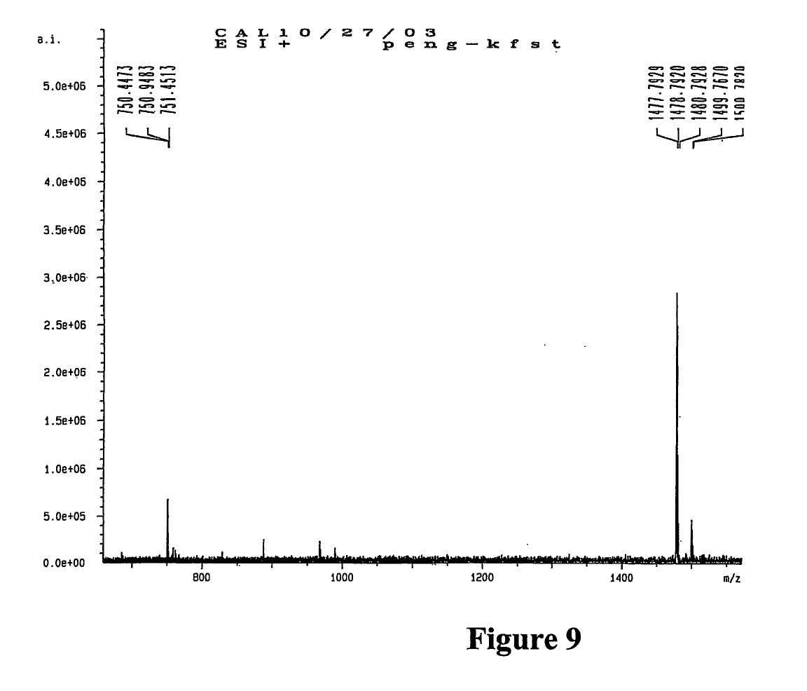

The combined physical data including comparable retention time by reversed phase HPLC, the relative intensities of the UV absorption spectra at 214 and 254 nm and the HRESI-MS data for the standard kahalalide F and the metabolite produced in culture confirms that the microbe identified as Vibrio sp. strains HV1 and HV10 are capable of producing kahalalide F. The high resolution mass spectral data shows a molecular ion at 1477.7929 for the cultured KF (FIG. 9) while the standard KF from Elysia shows a molecular ion (M+H⁺) at 1477.8304 (FIG. 6). Based on the half mass differences the signals at 750 represent a double charge species of KF where one of the charges is sodium and the other is a proton. This is a usual diagnostic indicator for KF since its ability to chelate positive ions is part of the proposed mechanism for drug action.

In a similar way, the pellet (2.76 g) of batch 110603 and the pellet (2.7 g) and supernatant (400 g) of batch 102903 were analyzed for KF and other kahalalides. KF was not found in pellets of either of the samples, but was found in the supernatant Vibrio sp strain HV10 in batch 102903.

While the invention has been described herein with reference to specific features, aspects and embodiments, it will be recognized that the invention may be widely varied, and that numerous other variations, modifications and other embodiments will readily suggest themselves to those of ordinary skill in the art. Accordingly, the ensuing claims are to be broadly construed, as encompassing all such other variations, modifications and other embodiments, within their spirit and scope.

### REFERENCES

All reference discussed herein are hereby incorporated by reference in their entirety and for all purposes.
1. Altschul, S. F., W. Gish, W. Miller, E. W. Myers, and D. J. Lipman. 1990. Basic local alignment search tool. Journal of Molecular Biology 215:403-410.
2. El Sayed, K. A., P. Bartyzel, X. Shen, T. L. Perry, J. K. Zjawiony, and M. T. Hamann. 2000. Marine natural products as antituberculosis agents. Tetrahedron 56:949-953.
3. Felsenstein, J. 2004. PHYLIP (Phylogenetic Inference Package), Version 3.6. Department of Genetics, University of Washington, Seattle, Wash.
4. Fitch, W. M., and E. Margoliash. 1967. Construction of phylogenetic trees: a method based on mutation distances as estimated from cytochrome c sequences is of general applicability. Science 155:279-284.
5. García-Rocha, M., P. Bonay, and J. Avila. 1996. The antitumoral compound Kahalalide F acts on cell lysosomes. Cancer Letters 99:43-50.
6. Hamann, M. T., C. S. Otto, P. J. Scheuer, and D. C. Dunbar. 1996. Kahalalides: bioactive peptides from a marine mollusk Elysia rufescens and its algal diet Bryopsis sp. Journal of Organic Chemistry 61:6594-6600.
7. Hamann, M. T., and P. J. Scheuer. 1993. Kahalalide F: A bioactive depsipeptide from the sacoglossan mollusk Elysia rufescens and the green alga Bryopsis sp. Journal of the American Chemical Society 115:5825-5826.
8. Jukes, T. H., and C. R. Cantor. 1969. Evolution of protein molecules, p. 21-132. In H. N. Munro (ed.), Mammalian protein metabolism. Academic Press, New York.
9. Kluge, A. G., and F. S. Farris. 1969. Quantitative phyletics and the evolution of annurans. Systematic Zoology 18:1-32.
10. Lopez-Macia, A., J. C. Jimenez, M. Royo, E. Giralt, and F. Albericio. 2000. Kahalalide B. Synthesis of a natural cyclodepsipeptide. Tetrahedron Letters 41:9765-9769.
11. Lopez-Macia, A., J. C. Jimenez, M. Royo, E. Giralt, and F. Albericio. 2001. Synthesis and structure determination of kahalalide F. Journal of the American Chemical Society 123:11398-11401.
12. Saitou, N., and M. Nei. 1987. The neighbor-joining method: a new method for reconstructing phylogenetic trees. Molecular Biology and Evolution 4:406-425.
13. Waterbury, J. B., and R Y. Stanier. 1978. Patterns of growth and development in pleurocapsalean cyanobacteria. Microbiological Reviews 42:2-44.
14. Webster, N. S., and R. T. Hill. 2001. The culturable microbial community of the Great Barrier Reef sponge Rhopaloeides odorabile is dominated by an α-Proteobacterium. Marine Biology 138:843-851.

That which is claimed is:

1. An isolated Vibrio sp. bacterium which produces a kahalalide.

2. The Vibrio sp. bacterium according to claim 1, wherein the Vibrio sp. bacterium is a V. mediterranei strain.

3. The Vibrio sp. bacterium of claim 1 where the kahalalide produced is kahalalide F.

4. An isolated Vibrio sp. bacterium which produces at least one kahalalide compound and which comprises a 16S rRNA having a nucleotide sequence of SEQ ID NO: 1 or SEQ ID NO: 2.

5. An isolated Vibrio sp. bacterium which produces at least one kahalalide compound and which comprises a 16S rRNA that hybridizes under high stringency conditions to SEQ ID NO: 1, SEQ ID NO: 2 or a complement thereof.

6. The isolated Vibrio sp. bacterium according to claim 2 comprising a 16S rRNA having a nucleotide sequence of SEQ ID NO: 1 or SEQ ID NO: 2.

7. The isolated Vibrio sp. bacterium according to claim 4, wherein the Vibrio sp. bacterium is a V. mediterranei strain.

8. The isolated Vibrio sp. bacterium according to claim 5, wherein the Vibrio sp. bacterium is V. mediterranei strain.

9. The isolated Vibro sp. bacteria according to claim 6, wherein the Vibro sp. bacterium is V. mediterranei strain.

10. The isolated Vibro sp. bacterium of claim 4, where the kahalalide produced is kahalalide F.

11. The isolated Vibro sp. bacterium of claim 5, where the kahalalide produced is kahalalide F.

12. The isolated Vibro sp. bacterium of claim 6, where the kahalalide produced is kahalalide F.

13. The isolated Vibro sp. bacterium according to claim 4, wherein the Vibro sp. bacterium is a strain HV1 or HV10 comprising a 16S rRNA of SEQ ID NO: 1 or SEQ ID NO: 2, respectively.

14. A method of isolating kahalalide-producing Vibro sp. bacteria comprising the steps of:
a) identifying bacteria comprising a 16S rRNA nucleotide sequence of SEQ ID NO: 1 or SEQ ID NO: 2;
b) screening bacteria for kahalalide producing ability; and
c) selecting those bacteria having kahalalide producing ability.

15. The method of claim 14, further comprising the step of screening bacteria to determine Vibro sp. bacteria morphology prior to step a).

16. A method of isolating kahalalide-producing Vibro sp. bacteria comprising the steps of:
a) identifying bacteria containing a 16S rRNA that hybridizes to SEQ ID NO: 1, SEQ ID NO: 2 or a complement thereof under high stringency conditions;
b) screening bacteria which hybridize in step a) for kahalalide producing ability, and
c) selecting those bacteria having kahalalide producing ability.

17. The method of claim 16, further comprising the step of screening bacteria to determine Vibro sp. bacteria morphology prior to step a).

18. An isolated polynucleotide comprising the sequence as set forth in SEQ ID NO: 1 or SEQ ID NO: 2.

19. An isolated polynucleotide as set forth in SEQ ID NO: 1 or SEQ ID NO: 2.

20. An isolated polynucleotide fragment comprising at least ten contiguous nucleotides of SEQ ID NO: 1 or SEQ ID NO: 2.

21. A method for producing kahalalide by fermentation, the method comprising:
a) culturing a Vibro sp. bacterium having kahalalide producing ability in a culture medium suitable for the growth of the Vibro sp. bacterium and production of kahalalide; and
b) separating the kahalalide from the culturing medium.

22. The method according to claim 21, wherein the culturing medium is maintained at a salinity in the range of about 15 ppt to about 25 ppt.

23. The method according to claim 21, wherein the Vibro sp. bacterium is V. mediterranei

24. The method according to claim 22, wherein the Vibrio sp. bacterium is strain HV1 or HV10 comprising a 16S rRNA nucleotide sequence of SEQ ID NO: 2 and SEQ ID NO: 3, respectively.

25. An isolated bacterium which produces a kahalalide compound.

26. The bacterium according to claim 25 comprising a 16S rRNA comprising a nucleotide sequence that hybridizes with SEQ ID NO: 1 or SEQ ID NO: 2 under high stringency conditions.

27. A method for detecting bacterium having kahalalide producing ability, the method comprising the steps of:
(a) mixing at least a fragment of a complement of the polynucleotide sequence of SEQ ID NO: 1 or SEQ ID NO: 2, with a biological test sample containing nucleic acids from a bacterium suspected of having kahalalide generating ability, to form a resulting mixture;
(b) subjecting the mixture to high or medium stringency conditions such that hybridization will occur between the biological test sample and the complement of the polynucleotide sequence of SEQ ID NO: 1 or SEQ ID NO: 2; and
(c) detecting hybridization complexes in the mixture subjected to hybridization conditions.
(d), wherein the presence of a hybridization complex correlates with the presence of a bacterium having kahalalide generating ability.

28. The method according to claim 27, further comprising screening of the bacterium having a nucleotide sequence that hybridized in step (c) for kahalalide generating ability.
